(19) United States
(12) Patent Application Publication
(10) Pub. No.: US 2007/0232508 A1
(43) Pub. Date: Oct. 4, 2007

(54) CREAM-TYPE SKIN CLEANSING COMPOSITION

(75) Inventor: Eiko OSHIMURA, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Kawasaki-shi (JP)

(21) Appl. No.: 11/694,055

(22) Filed: Mar. 30, 2007

(30) Foreign Application Priority Data
Mar. 30, 2006 (JP) 2006-095878

(51) Int. Cl. A61K 8/00; A61Q 19/00

(52) U.S. Cl. 510/130; 424/70.1

(57) ABSTRACT

A cream-type skin cleansing composition containing substantially no polyhydric alcohol, which weakly acidic with a pH of 4.0 to 6.0, and soft and smooth with a whity appearance is provided, which is excellent in foamability as well as rinsability and in feeling during application, but avoids from becoming hard at low temperatures, and which can be readily prepared by using a common simple mixer, which contains an N-long-chain acylglycine or a salt thereof, a polyoxyethylene alkyl ether sulfate salt, a betaine type amphoteric surfactant, an inorganic or organic salt and water at a specified amounts and pH.

### FIELD OF THE INVENTION

The present invention relates to a cream-type skin cleansing composition comprising an N-long-chain acylglycine and/or a salt thereof, a polyoxyethylene alkyl ether sulfate salt, a betaine type amphoteric surfactant, an inorganic salt and/or salt of pyrrolidone carboxylate and water, where the mixing ratio and the pH of the composition are as specified herein. The present invention also relates to a cream-type cleansing composition further comprising a fatty acid and/or alkylpolyglucoside.

### BACKGROUND OF THE INVENTION

N-long-chain acylglycines and/or the salts thereof have been extensively applied to skin cleansing preparations as a mild cleansing agent that is excellent in foamability and in rinsability. As for facial cleansing applications, cream-type skin cleansing formulations have been preferred in particular for the following reasons: convenience achieved by usability of taking out an appropriate amount from the tube as needed and by easy dissolution in water, neatness provided by possible avoidance of deformation by dissolution that occurs in bar soap and of staining around wash basins due to dripping of liquid cleansing formulations. Hence, various investigations have been performed for producing cream-type compositions using N-long-chain acylglycine and/or salts thereof.

Japanese Patent No. 3304646 proposes a process for obtaining a cream-type formulation that is excellent in foaming and rinsing properties by blending N-long-chain acylglycine or a salt thereof, an inorganic salt, and polyalcohol. However, with this composition, polyalcohol is liable to be separated, and thus creams could not be obtained with favorable reproducibility.

JP-A No. 2000-143497 discloses a process for obtaining cream-type compositions that are excellent in form stability by using an N-long-chain acyl neutral amino acid or a salt thereof, particularly an N-long-chain acylglycine salt, and polyalcohol and a highly polymerized polyol. However, with respect to these compositions, in order to obtain soft cream, after heating all the components to be melted, it is necessary to provide enough stirring during cooling process. In this process, a phenomenon of air bubble entrainment (a phenomenon of inclusion of air bubble, which remains in a product, due to sudden increase in viscosity) is caused, resulting in deterioration of both appearance and smoothness of the cream. Additionally, odorization as well as hardening of the cream during storage at low temperatures was also caused by compounding polyalcohol in such large quantity of 5% by weight or more.

JP-A No. 2001-19632 proposes a process in which an N-long-chain acylglycine alkali salt, a betaine amphoteric surfactant, water and an organic acid are used for proving the problem of hardening of the cream during storage at low temperatures. Also, JP-A No. 2002-265352 discloses a process in which an N-long-chain acylamino acid salt, an imidazoline type amphoteric surfactant, a water-swelling clay mineral, and monoalkyl fatty acid glyceryl are used. However, in both descriptions, deterioration of the appearance due to the air bubble entrainment during the production is not yet prevented, but another problems of decrease in foamability and rinsability could be caused, which may result from a great proportion of cationic type of the imidazoline amphoteric surfactant or the betaine amphoteric surfactant, in a weakly acidic region of the pH not higher than 6.0.

JP-A No. 2002-363061 proposes a process in which an N-long-chain acylamino acid salt, silicic anhydride and water soluble polyalkylene glycol are used in combination, as another means for improving stability of the cream. However, when an acylglycine salt is used as the N-long-chain acylamino acid salt, silicic anhydride is likely to founder, leading to heterogeneity of the cream.

U.S. Pat. No. 477,424 proposes a process of providing a highly viscous composition by incorporating a long-chain acyl sarcosine salt, a polyoxyethylene alkyl ether sulfate salt, and a betaine amphoteric surfactant in combination at a specific mixing ratio. However, those compositions were developed anticipating use as shampoo, which are provided in the form of a liquid cleansing formulation with markedly high viscosity. Therefore, in an attempt to use it as a creamy cleansing formulation, some difficulties may be involved in scooping with a finger and in blending with water on the palm.

Cream-type skin cleansing compositions containing substantially no polyalcohol, weakly acidic with a pH of 4.0 to 6.0 were strongly desired. Other requirements include a soft and smooth texture with a whity appearance, excellent in foamability and rinsability as well as in feeling during application such as moisturizing and fresh dry-skin feel, but can avoid becoming hard at low temperatures, and which can be readily prepared by using a common simple mixer.

### SUMMARY OF THE INVENTION

An object of the present invention is to provide a cream-type skin cleansing composition which contains substantially no polyhydric alcohol, is weakly acidic with a pH of 4.0 to 6.0, and has a soft and smooth texture with a whity appearance, which is excellent in foamability and rinsability as well as in feeling during application such as moisturizing and fresh dry-skin feel, but can avoid becoming hard at low temperatures, and which can be readily prepared by using a common simple mixer.

The present inventor conducted studies in order to solve the foregoing problems, and consequently found that the problems can be solved by allowing N-long-chain acylglycine or a salt thereof, a polyoxyethylene alkyl ether sulfate salt, a betaine type amphoteric surfactant, an inorganic and/or pyrrolidone carboxylate salt and water to be included at a specific compounding ratio and pH. Hence, the present invention was accomplished.

Accordingly, the present invention provides the following embodiments.

In one embodiment, the present invention provides a cream-type skin cleansing composition which includes N-long-chain acylglycine and/or a salt thereof (component A), a polyoxyethylene alkyl ether sulfate salt (component B), a betaine type amphoteric surfactant (component C), an inorganic salt and/or a pyrrolidone carboxylate salt (component D), and water (component E), wherein the composition is characterized by: [total weight of the (component A), (component B) and (component C)] accounting for 12 to 30% by weight of total weight of the composition; [weight of the (component A)]/[total weight of the (component A), (component B) and (component C)] being 3/8 to 4/5; [weight of the (component B)]/[weight of the (component C)] being 9/1 to 1/1; and the pH being 4.0 to 6.0.

In another embodiment, the present invention also provides the cream-type skin cleansing composition characterized by further including a higher fatty acid (component F) and/or alkylpolyglucoside (component G) in an amount of 0.01 to 5.0% by weight, in addition to the components described above.

A cream-type skin cleansing composition containing substantially no polyhydric alcohol, being weakly acidic with a pH of 4.0 to 6.0, and having soft and smooth texture with a whity appearance can be obtained, which is excellent in foamability and rinsability as well as in sensory feeling such as moisturizing and fresh dry-skin feel, but can avoid from getting hard at low temperatures, and which can be readily prepared by using a common simple mixer.

### DETAILED DESCRIPTION OF THE INVENTION

In the cream-type skin cleansing composition of the invention, N-long-chain acylglycine and/or a salt thereof (component A), a polyoxyethylene alkyl ether sulfate salt (component B), a betaine type amphoteric surfactant (component C), an inorganic salt and/or a pyrrolidone carboxylate salt (component D), and water (component E) are blended in a specific ratio. The term “cream” as used herein means a form having an appearance like hand cream that is whity, soft, smooth and shiny, and being readily scooped with a finger when filled in a tube or jar without causing dripping and marked stringing. Hereinafter, the constitutive components and compounding ratio will be sequentially explained.

As the long-chain acyl group of the long-chain N-acylglycine and/or the salt thereof (component A) for use in the invention, any one derived from saturated or unsaturated fatty acid having a straight or branched chain with 8 to 22 carbon atoms can be used. In the case of acyl groups with less than 8 carbon atoms, foamability and cleansing properties may be insufficient as the cleansing agent. To the contrary, in the case in which more than 22 carbon atoms are present, solubility in the cleansing composition may be deteriorated to yield the composition with impaired smoothness. Examples of the fatty acid referred to herein include caprylic acid, capric acid, lauric acid, myristic acid, stearic acid, isostearic acid, palmitic acid, oleic acid, linoleic acid, behenic acid, coconut oil fatty acid, palm fatty acid, hydrogenated beef tallow fatty acid, and the like. These may be used alone, or 2 or more thereof may be used as a mixture. In particular, in light of favorable foamability and foam quality, coconut oil fatty acid, lauric acid, and myristic acid are preferred. The N-acylglycine can be produced according to a known process such as subjecting to a Schotten-Baumann reaction with glycine under a condition of an aqueous alkali solution, and the like, after converting the fatty acid into fatty acid chloride by common procedures.

The salt of the (component A) which may be used in the invention is not particularly limited, but specific examples include alkali metal salts such as sodium and potassium, alkaline earth metal salts such as magnesium, organic amine salts such as ammonium, diethanolamine and triethanolamine, basic amino acid salts such as lysine and arginine. In light of avoiding deterioration of the form resulting from unexpected salt exchange with other ionic component, alkali metal salts are preferred, and sodium or potassium is particularly preferred.

The polyoxyethylene alkyl ether sulfate salt (component B) for use in the invention is not particularly limited, but a lauryl ether sulfate salt is preferred, and a polyoxyethylene alkyl ether sulfate salts which are represented by the following general formula (1) and which meet specific requirements are more preferred, and those in which n is 2 to 3 mol are particularly preferred.

R—(OC₂H₄)ₙ—OSO₃M (1)

wherein, R represents an alkyl group having 8 to 24 carbon atoms; n represents an average number of added moles of ethylene oxide, and may be 0.5≦n≦8; and M represents a metal, organic amine, or basic amino acid.

The salt (M) of the (component B) used in the invention is not particularly limited, however, an alkali metal salt such as sodium or potassium, an alkaline earth metal salt such as magnesium, an organic amine salt such as ammonium, diethanolamine or triethanolamine, a basic amino acid salt such as lysine or arginine, or the like can be used. These may be used alone, or 2 or more thereof may be used as a mixture. In light of favorable foamability and cleansing power, alkali metal salts are preferred, and sodium salts are particularly preferred.

Although the betaine type amphoteric surfactant (component C) used in the invention is not particularly limited, specifically, an acetic betaine type amphoteric surfactant, an amide betaine type amphoteric surfactant, a sulfo betaine type amphoteric surfactant, a phospho betaine type amphoteric surfactant, or an imidazolinium betaine type amphoteric surfactant can be used. Preferred examples of the betaine type amphoteric surfactant include amide betaine type amphoteric surfactants represented by the following general formula (2). Preferred examples of the amide betaine type amphoteric surfactant include lauric amide propyl betaine, coconut oil fatty amide propyl betaine, and myristic amide propyl betaine. In light of foamability, and stability of the composition, coconut oil fatty amide propyl betaine is particularly preferred.

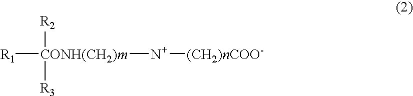

wherein R¹ represents a straight or branched alkyl or alkenyl group having 5 to 21 carbon atoms; R², R³ represents a hydrogen atom, or a straight or branched alkyl group having 1 to 3 carbon atoms; n represents an integer of 1 to 3; and m represents an integer of 1 to 4.

Although the inorganic salt or pyrrolidone carboxylate salt of the (component D) used in the invention is not particularly limited, any one produced by a neutralizing reaction of an acid with a base can be generally used. Examples of the acid include inorganic acids such as sulfuric acid, hydrochloric acid, phosphoric acid and carbonic acid; acidic amino acids such as glutamic acid and pyrrolidone carboxylic acid; organic acids such as citric acid and gluconic acid, and the like. Examples of the base include alkali metals such as sodium and potassium, alkaline earth metals such as magnesium and calcium, basic amino acids such as lysine, arginine and ornithine, organic amines such as ammonia, triethanolamine, diethanolamine, and the like. The basic amino acid or pyrrolidone carboxylic acid which may be used is either one of a racemic body, or an optically active substance. Specifically, sodium chloride, potassium chloride, magnesium chloride, calcium chloride, sodium sulfate, sodium hydrogen sulfate, potassium sulfate, potassium hydrogen sulfate, magnesium sulfate, calcium sulfate, monosodium phosphate, disodium phosphate, monopotassium phosphate, dipotassium phosphate, sodium carbonate, disodium hydrogen carbonate, potassium carbonate, potassium hydrogen carbonate, magnesium carbonate, calcium carbonate, sodium pyrrolidone carboxylate, sodium L-pyrrolidone carboxylate, potassium DL-pyrrolidone carboxylate, ammonium DL-pyrrolidone carboxylate, or the like can be used. These salts may be used alone, or 2 or more thereof may be used as a mixture. In light of possibility of obtaining the cream that is smooth and whity and has a favorable form, sodium chloride, potassium chloride, sodium DL-pyrrolidone carboxylate, sodium L-pyrrolidone carboxylate, potassium DL-pyrrolidone carboxylate are preferred, and sodium chloride and sodium pyrrolidone carboxylate are more preferred.

Water which is the (component E) used in the invention is not particularly limited as long as it has a purity to the extent generally employed for cleansing agents and cosmetics. Specifically, ion exchanged water, well water, natural water, ground water, city water, hard water, soft water or the like can be used. These may be used alone, or 2 or more thereof may be used as a mixture. In light of storage stability and health aspects of the present product, ion exchanged water is preferred.

The pH of the cream-type skin cleansing composition of the invention may be selected to fall within the range of from 4.0 to 6.0. When the pH is lower than 4.0, deterioration of foamability may be caused due to increase in unneutralized acylglycine. To the contrary, when the pH is higher than 6.0, deformation of the creamy state may occur because solubility of the acylglycine salt in the composition is elevated. In light of possible formation of the creamy state in a stable manner, the pH is preferably 4.5 to 6.0, and more preferably 5.3 to 5.8. The acid for adjusting the pH is not particularly limited, but specific examples include organic acids such as citric acid, lactic acid, acetic acid, malic acid, tartaric acid and glycol acid; acidic amino acids such as aspartic acid, glutamic acid and pyrrolidone carboxylic acid; inorganic acids such as hydrochloric acid, sulfuric acid and phosphoric acid, and the like. In light of accompanying pH buffering capacity, thereby capable of providing the pH hardly altered by other component, citric acid, tartaric acid, glutamic acid and phosphoric acid are preferred; citric acid and tartaric acid are more preferred; and citric acid is particularly preferred.

Total weight of the (component A), (component B) and (component C) of the invention is employed to fall within the range of 12 to 30% by weight per total weight of the composition. When it is less than 12% by weight, the cream may be heterogeneous, while when it is beyond 30% by weight, the composition becomes so hard that the creamy state cannot be maintained. In light of being able to keep the creamy state in a stable manner, it is preferably 15 to 28% by weight, and more preferably 16 to 25%.

The ratio of [weight of the (component A)]/[total weight of the (component A), (component B) and (component C)] of the invention is employed to fall within the range of 3/8 to 4/5. When the ratio is less than 3/8, heterogeneous form is liable to be yielded through partial dissolution of the acylglycine salt, while when the ratio is beyond 4/5, the composition is liable to be hard. In both cases, deterioration of the creamy state is caused. Particularly, in light of possible achievement of excellent foamability, 2/5 to 3/4 is preferred; 5/12 to 5/7 is more preferred, and 5/12 to 2/3 is particularly preferred.

The ratio [weight of the (component B)]/[weight of the (component C)] of the invention is selected to fall within the range of 1/1 to 9/1. When the ratio is less than 1/1, the cream accompanied by inferior handling performance with potent stringiness can be prepared, while when the ratio is beyond 9/1, the composition is likely to have a heterogeneous form. In light of capability of obtaining the cream having preferable form, the ratio preferably falls within the range of 1/1 to 9/1, more preferably in the range of 2/1 to 8/1, and particularly preferably in the range of 3/1 to 5/1.

Percentage of the weight of the (component D) weight/total weight of the composition of the invention is employed to fall within the range of 0.1 to 5% by weight. When the percentage is less than 0.1% by weight, the cream may loose whiteness during storage at high temperatures to be transparent, while when the percentage is beyond 5% by weight, the composition may cause separation during storage at high temperatures. In light of effect of imparting stable whiteness exhibited, the percentage of 0.2 to 3% by weight is preferred, 0.3 to 2.5% by weight is more preferred, and 0.5 to 2.0% by weight is particularly preferred.

Each characteristic can be imparted by further blending the fatty acid (component F) and alkylpolyglucoside (component G) in the cream-type skin cleansing composition including the (component A) to (component E) and having a specific compounding ratio and pH according to the invention. The (component F) and the (component G) may be used in combination.

The cream-type skin cleansing composition including the (component A) to (component E) and having a specific compounding ratio and pH according to the invention can have increased whiteness and can be even more soft when it is stored at low temperatures by further adding the fatty acid (component F). Examples of the fatty acid salt which may be used include saturated or unsaturated fatty acids having a straight or branched chain with 8 to 22 carbon atoms. Specific examples thereof include caprylic acid, capric acid, lauric acid, myristic acid, stearic acid, isostearic acid, palmitic acid, oleic acid, linoleic acid, behenic acid, coconut oil fatty acid, palm fatty acid, hydrogenated beef tallow fatty acid, and the like. These may be used alone, or 2 or more thereof may be used as a mixture. In particular, in light of favorable foamability and foam quality, coconut oil fatty acid, lauric acid, and myristic acid are preferred.

The amount of the included (component F) of the invention is not particularly limited, but it is preferably used in an amount of 0.01 to 5% by weight per the weight of the composition including the (component A) to the (component E). When the amount is less than 0.01% by weight, effect of imparting whiteness to the cream may not be achieved, while when the amount is beyond 5% by weight, viscosity and foamability of the cream may be deteriorated. In light of capability of efficacious achievement of the increase in whiteness, and further improvement of the softness during storage at low temperatures of the cream, the amount is preferably 0.1 to 3% by weight, and more preferably 0.2 to 2% by weight.

Stability during storage at high temperatures can be even more improved by further adding alkylpolyglucoside as the (component G) to the cream-type skin cleansing composition including the (component A) to (component E) and having a specific compounding ratio and pH according to the invention. Although the alkylpolyglucoside is not particularly limited, any one represented by the following general formula (3) is more preferred.

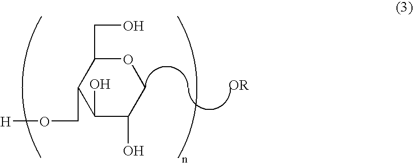

wherein, R represents an alkyl group having 8 to 24 carbon atoms; n represents an average number of polymerized moles of glucose, and may be 1≦n≦3. Specific examples include caprylyl glucoside, decyl glucoside, lauryl glucoside, myristyl glucoside, undecyl glucoside, coconut oil alkyl glucoside, cetearyl glucoside, isostearyl glucoside and the like. These may be used alone, or 2 or more thereof may be used as a mixture. Particularly, in light of excellent effect to improve the storage stability at high temperatures, lauryl glucoside, palm coconut oil alkyl glucoside are preferred.

The amount of the included (component G) of the invention is not particularly limited, but it is preferably used in an amount of 0.01 to 5% by weight per the weight of the composition including the (component A) to the (component E). When the amount is less than 0.01% by weight, the effect to improve the stability at high temperatures may not be sufficient, while when the amount is beyond 5% by weight, foamability of the cream may be deteriorated. In light of capability of distinct achievement of the effect, the amount is preferably 0.1 to 3% by weight, and more preferably 0.2 to 2% by weight.

To the cream-type cleansing composition of the invention, in addition to the aforementioned components, can be added various additives generally used in cosmetics in the range not to impair the effect of the invention. The additive can be selected ad libitum by persons skilled in the art depending on the desired characteristics, and type thereof is not particularly limited, but examples thereof include e.g., oily materials for cosmetics, silicone compounds, organic salts, hydrolyzed proteins, alcohols, extracts, amino acids, vitamins, enzymes, anti-inflammatory agents, bacteriocides, antiseptic agents, anti-oxidizing agents, ultraviolet ray absorbing agents, chelating agents, antiperspirants, pigments, dye stuffs, oxidized dyes, pearlescent aids, wetting agents, moisturizing agents, surfactants, inorganic powder, organic powder, water soluble polymers, and the like.

The cream-type cleansing composition of the invention can be used as any of various cleansing agents and cosmetics. Specific examples of the application include beauty soaps, facial washes (creamy or paste form, liquid or gel form, with use of aerosol and the like), cleaning cosmetics such as shampoo, creams for shaving (after shaving cream, shaving cream and the like), oral cosmetics such as toothpaste, and the like. In light of excellent feel in use, facial washes, creams for shaving, and oral cosmetics are preferred.

### EXAMPLES

Hereinafter, the present invention will be explained in more detail by way of Examples, but the invention is not limited to these Examples.

### Evaluation Method

Hereinafter, evaluation methods will be sequentially explained.

### Form

The composition was left to stand at a room temperature, and categorized according to the following criteria based on results of visual inspection and feeling experienced in touch with a finger. In the assessment, those categorized in P were assigned as A; S or V as B; and L (liquid) or SP (separation) as C.
P: being white with almost no fluidity, but readily flows upon pushing with a finger;
S: being white exhibiting a block with completely no fluidity, but is deformed upon pushing with a finger;
V: being a transparent or white turbid liquid having a viscosity higher than a level to permit filling in a tube;
L (liquid): being a transparent or white turbid liquid not having a viscosity of a level to permit filling in a tube SP (separation): being separated into liquid part and solid part, or two or more phases being present having apparently distinct states.

### Whiteness

Whiteness of the composition was visually discriminated based on the following criteria of assessment.
A: having strong whiteness entirely, without unevenness, and being glossy;
B: having entirely whiteness;
C: having low whiteness with transparency, or being slightly colored;
D: corresponding to any one of having no whiteness, being transparent, having unevenness of the color, being strongly colored.

### Softness

Sensory evaluation was conducted by five panelists through placing the composition on a palm, spreading over with a finger of another hand, and categorizing into:
4: being very soft;
3: being soft;
2: being moderate in softness;
1: not being somewhat soft; and
0: not being soft.

Results were represented based on the average point, i.e., A: 3.1 or higher; B: 2.5 to 3.0; C, 2.0 to 2.4; and D: 1.9 or lower.

### Softness (0° C.)

Sensory evaluation was conducted by five panelists through placing the composition on a palm, immediately after removing from a storage cabinet following leaving the composition to stand in the storage cabinet at 0° C. for 7 days, spreading over with a finger of another hand, and categorizing into:
4: being very soft;
3: being soft;
2: being moderate in softness;
1: not being somewhat soft; and
0: not being soft.

Results were represented based on the average point, i.e., A: 3.1 or higher; B: 2.5 to 3.0; C, 2.0 to 2.4; and D: 1.9 or lower.

### Smoothness

Sensory evaluation was conducted by five panelists through placing the composition on a palm, spreading over with a finger of another hand, and categorizing the texture into:
4: being very smooth;
3: being smooth;
2: being moderate in smoothness;
1: being somewhat deficient in smoothness; and
0: being deficient in smoothness, or not being homogenous, or including grains.

Results were represented based on the average point, i.e., A: 3.1 or higher; B: 2.5 to 3.0; C, 2.0 to 2.4; and D: 1.9 or lower.

### Break

Sensory evaluation was conducted by five panelists through placing the composition on a palm, scooping a part thereof with a finger of another hand, and categorizing into:
4: being extremely excellent in break, not stringy, not dripping;
3: being excellent in break;
2: being moderate in break;
1: not being preferred because of somewhat inferior break such as stringing; and
0: not being preferred because of inferior break, or dripping.

Results were represented based on the average point, i.e., A: 3.1 or higher; B: 2.5 to 3.0; C, 2.0 to 2.4; and D: 1.9 or lower.

### Foamability

Sensory evaluation was conducted by five panelists through placing 0.5 g of the composition on a palm, adding small quantity of water, followed by allowing foaming for 30 seconds while rubbing with another hand, and categorizing the foamability into:
4: being very favorable;
3: being favorable;
2: being moderate;
1: being somewhat insufficient; and
0: being insufficient.

Results were represented based on the average point, i.e., A: 3.1 or higher; B: 2.5 to 3.0; C, 2.0 to 2.4; and D: 1.9 or lower.

### Rinsability

Sensory evaluation was conducted by five panelists through allowing foaming 0.5 g of the composition with both hands, rinsing with running water at 35° C. to 40° C., and categorizing the rinsability into:
4: being very rapid;
3: being rapid;
2: being moderate;
1: being somewhat slow; and
0: being slow.

Results were represented based on the average point, i.e., A: 3.1 or higher; B: 2.5 to 3.0; C, 2.0 to 2.4; and D: 1.9 or lower.

### Odor

Sensory evaluation was conducted by five panelists, and categorizing the odor into:
4: completely odor-free;
3: almost odor-free;
2: yielding slight unpleasant odor felt;
1: yielding definitive unpleasant odor felt; and
0: yielding strong unpleasant odor felt.

Results were represented based on the average point, i.e., A: 3.1 or higher; B: 2.5 to 3.0; C, 2.0 to 2.4; and D: 1.9 or lower.

### Blendability with Water

Sensory evaluation was conducted by five panelists through placing the composition on a palm, adding small quantity of water, followed by rubbing with another hand, and categorizing the time period until the form of the composition is lost in dissolution into:
4: being very short;
3: being somewhat short;
2: being moderate;
1: not being somewhat preferred because of a long time period required; and
0: not being preferred because of an extremely long time period required.

Results were represented based on the average point derived from the score list, A: 3.1 or higher; B: 2.5 to 3.0; C, 2.0 to 2.4; and D: 1.9 or lower.

### Air Bubble Entrainment Height

The composition in an amount of 150 g was placed in a vessel of avacuum emulsifier (manufactured by Tokushu Kika Kogyo Co., Ltd., T. K. AGI HOMO MIXER® 2M-03), and completely melted with the circulating water at a temperature of 85° C. to give the temperature of the composition of approximately 65° C. Cooling was started by lowering the temperature of the circulating water at a rate of 10° C./10 min while stirring with a rotation speed of the puddle (agitation propeller) of 20 rpm, and a rotation speed of scraping of 30 rpm. The agitation was terminated after 30 min from the time point when the composition started to get turbid white, and the surface of the composition in the vessel was made flat. The height of the surface of the composition from the bottom face of the vessel was measured with a ruler to determine the air bubble entrainment height. On the basis of the height of 3.5 cm when 150 g of water was charged in the vessel, the composition height of not higher than 3.4 cm was assigned as A; 3.5 cm to 5.2 cm as B; the composition height of 5.3 cm to 6.9 cm as C; and the composition height of 7.0 cm or higher as D.

### Hardness

The composition in an amount of 50 g was placed in a glass vial having an internal diameter of 3.5 cm, and a height of 5.3 cm, and the vial was left to stand in a room at 25° C. overnight. Maximum force measured when a stainless cylinder having a diameter of 1.5 cm was entered with a rheometer (manufactured by Fudo Kogyo K K, NRM-2010J-CW) at a rate of 6 cm/min for 30 sec was determined as the hardness. The hardness of less than 50 g was assigned as A; 50 g to 100 g as B; 100 to 500 g as C; and 500 g or higher as D.

### Stability at High Temperature

With respect to the stability at a high temperature, these compositions were filled in a tube having an outlet diameter of 5 mm, and left to stand in a storage cabinet at 45° C. for 7 days. The composition immediately after removing from the storage cabinet was visually discriminated based on the following criteria.

A: the composition does not flow out even though the outlet of the tube is brought downward, with almost no fluidity on the palm;

B: the composition does not flow out even though the outlet of the tube is brought downward, but flows very slowly on the palm;

C: the composition does not flow out even though the outlet of the tube is brought downward, but flows on the palm;

D: the composition flows out when the outlet of the tube is brought downward.

### Examples 1 to 27, Comparative Examples 1 to 54

Cleansing compositions (Examples 1 to 27, Comparative Examples 1 to 54) having the composition shown in Table 1 below were prepared, and tested according to the following procedures. The amount of each compounded component shown in the Table (values in Table) represents the weight fraction (%) provided that total composition accounts for 100. Also, citric acid monohydrate was used each in an amount required for adjusting the pH of the composition. Procedure

1. Primary Evaluation: Evaluation of Composition Form (Form) was evaluated.

2. Secondary Evaluation: Evaluation of Quality of Composition as Cream

### The compositions determined as A or B in the primary evaluation were evaluated on the (Whiteness), (Softness), (Smoothness), (Break), and (Softness (0° C.)).

3. Tertiary Evaluation

### Evaluation on the (Foamability) and (Rinsability) was made for the compositions except for those categorized into D on two or more items in the secondary evaluation.

TABLE 1

| | | Example 1 | Example 2 | Example 3 | Example 4 | Example 5 | Example 6 | Example 7 | Example 8 | Example 9 |
|---|---|---|---|---|---|---|---|---|---|---|
| Component A: | sodium cocoyl glycinate | 7.50 | 7.50 | 7.50 | 9.00 | 9.00 | 9.00 | 12.00 | 12.00 | 12.00 |
| Component B: | sodium polyoxyethylene | 8.75 | 7.00 | 5.25 | 7.50 | 6.00 | 4.50 | 5.00 | 4.00 | 3.00 |
| | lauryl ether sulfate | | | | | | | | | |
| Component C: | cocamidopropyl betaine | 1.75 | 3.50 | 5.25 | 1.50 | 3.00 | 4.50 | 1.00 | 2.00 | 3.00 |
| Component D: | sodium chloride | 0.5 | 0.5 | 0.5 | 0.5 | 0.5 | 0.5 | 0.5 | 0.5 | 0.5 |
| | citric acid monohydrate | pH 6.0 | pH 6.0 | pH 6.0 | pH 6.0 | pH 6.0 | pH 6.0 | pH 6.0 | pH 6.0 | pH 6.0 |
| Component E: | water | balance | balance | balance | balance | balance | balance | balance | balance | balance |
| | Total | 100.0 | 100.0 | 100.0 | 100.0 | 100.0 | 100.0 | 100.0 | 100.0 | 100.0 |

| Component A/(Component A + Component | | 5/12 | 5/12 | 5/12 | 1/2 | 1/2 | 1/2 | 2/3 | 2/3 | 2/3 |
|---|---|---|---|---|---|---|---|---|---|---|
| B + Component C) | | | | | | | | | | |
| Component B/Component C | | 5/1 | 2/1 | 1/1 | 5/1 | 2/1 | 1/1 | 5/1 | 2/1 | 1/1 |
| Primary Evaluation: | Form | P | P | P | P | P | P | P | P | P |
| | Decision | A | A | A | A | A | A | A | A | A |
| Secondary Evaluation: | Whiteness | B | B | B | B | B | B | B | A | A |
| | Softness | A | A | A | A | A | A | A | A | A |
| | Smoothness | A | A | A | A | A | A | A | A | A |
| | Break | A | A | B | A | A | B | A | A | B |
| | Softness (0° C.) | B | A | A | B | A | A | B | A | A |
| Tertiary Evaluation: | Foamability | A | A | A | A | A | B | A | B | B |
| | Rinsability | A | A | B | A | A | A | A | A | B |

| | | Comparative | Comparative | Comparative | Comparative | Comparative | Comparative | Comparative | Comparative | Comparative | Comparative | Comparative | Comparative |
|---|---|---|---|---|---|---|---|---|---|---|---|---|---|
| | | Example 1 | Example 2 | Example 3 | Example 4 | Example 5 | Example 6 | Example 7 | Example 8 | Example 9 | Example 10 | Example 11 | Example 12 |
| Component A: | sodium cocoyl | 7.50 | 7.50 | 7.50 | 15.00 | 15.00 | 15.00 | 7.50 | 7.50 | 9.00 | 9.00 | 12.00 | 12.00 |
| | glycinate | | | | | | | | | | | | |
| Component B: | sodium | 8.75 | 7.00 | 5.25 | 2.50 | 2.00 | 1.50 | 10.50 | 3.50 | 9.00 | 3.00 | 6.00 | 2.00 |
| | polyoxyethylene | | | | | | | | | | | | |
| | lauryl ether sulfate | | | | | | | | | | | | |
| Component C: | cocamidopropyl | 1.75 | 3.50 | 5.25 | 0.50 | 1.00 | 1.50 | 0.00 | 7.00 | 0.00 | 6.00 | 0.00 | 4.00 |
| | betaine | | | | | | | | | | | | |
| Component D: | sodium chloride | 0.5 | 0.5 | 0.5 | 0.5 | 0.5 | 0.5 | 0.5 | 0.5 | 0.5 | 0.5 | 0.5 | 0.5 |
| | citric acid | pH 6.0 | pH 6.0 | pH 6.0 | pH 6.0 | pH 6.0 | pH 6.0 | pH 6.0 | pH 6.0 | pH 6.0 | pH 6.0 | pH 6.0 | pH 6.0 |
| | monohydrate | | | | | | | | | | | | |
| Component E: | water | balance | balance r | balance | balance | balance | balance | balance | balance | balance | balance | balance | balance |
| | Total | 100.0 | 100.0 | 100.0 | 100.0 | 100.0 | 100.0 | 100.0 | 100.0 | 100.0 | 100.0 | 100.0 | 100.0 |

| Component A/(Component A + Component | | 1/3 | 1/3 | 1/3 | 5/6 | 5/6 | 5/6 | 5/12 | 5/12 | 1/2 | 1/2 | 2/3 | 2/3 |
|---|---|---|---|---|---|---|---|---|---|---|---|---|---|
| B + Component C) | | | | | | | | | | | | | |
| Component B/Component C | | 5/1 | 2/1 | 1/1 | 5/1 | 2/1 | 1/1 | 1/0 | 1/2 | 1/0 | 1/2 | 1/0 | 1/2 |
| Primary | Form | L | V | V | P | P | P | L | P | L | P | P | P |
| Evaluation: | Decision | C | B | B | A | A | A | C | A | C | A | A | A |
| Secondary | Whiteness | — | C | C | A | A | A | — | B | — | B | C | B |
| Evaluation: | Softness | — | B | B | C | C | C | — | A | — | A | B | A |
| | Smoothness | — | D | C | C | C | C | — | B | — | D | D | B |
| | Break | — | C | D | A | A | A | — | C | — | C | C | C |
| | Softness (0° C.) | — | D | D | D | D | D | — | A | — | A | B | A |
| Tertiary | Foamability | — | — | — | D | D | D | — | D | — | D | B | D |
| Evaluation: | Rinsability | — | — | — | A | A | B | — | D | — | D | D | D |

| | | Example 10 | Example 11 | Example 12 | Example 13 | Example 14 | Example 15 | Example 16 | Example 17 | Example 18 |
|---|---|---|---|---|---|---|---|---|---|---|
| Component A: | sodium cocoyl glycinate | 7.50 | 7.50 | 7.50 | 9.00 | 9.00 | 9.00 | 12.00 | 12.00 | 12.00 |
| Component B: | sodium polyoxyethylene | 8.75 | 7.00 | 5.25 | 7.50 | 6.00 | 4.50 | 5.00 | 4.00 | 3.00 |
| | lauryl ether sulfate | | | | | | | | | |
| Component C: | cocamidopropyl betaine | 1.75 | 3.50 | 5.25 | 1.50 | 3.00 | 4.50 | 1.00 | 2.00 | 3.00 |
| Component D: | sodium chloride | 0.5 | 0.5 | 0.5 | 0.5 | 0.5 | 0.5 | 0.5 | 0.5 | 0.5 |
| | citric acid monohydrate | pH 5.5 | pH 5.5 | pH 5.5 | pH 5.5 | pH 5.5 | pH 5.5 | pH 5.5 | pH 5.5 | pH 5.5 |
| Component E: | water | balance | balance | balance | balance | balance | balance | balance | balance | balance |
| | Total | 100.0 | 100.0 | 100.0 | 100.0 | 100.0 | 100.0 | 100.0 | 100.0 | 100.0 |

| Component A/(Component A + Component | | 5/12 | 5/12 | 5/12 | 1/2 | 1/2 | 1/2 | 2/3 | 2/3 | 2/3 |
|---|---|---|---|---|---|---|---|---|---|---|
| B +Component C) | | | | | | | | | | |
| Component B/Component C | | 5/1 | 2/1 | 1/1 | 5/1 | 2/1 | 1/1 | 5/1 | 2/1 | 1/1 |
| Primary Evaluation: | Form | P | P | P | P | P | P | P | P | P |
| | Decision | A | A | A | A | A | A | A | A | A |
| Secondary Evaluation: | Whiteness | A | A | B | A | A | A | B | A | A |
| | Softness | A | A | A | A | A | A | A | A | A |
| | Smoothness | A | A | A | A | A | A | A | A | A |
| | Break | A | A | B | A | A | B | A | A | B |
| | Softness (0° C.) | A | A | A | A | A | A | A | A | A |
| Tertiary Evaluation: | Foamability | A | A | A | A | A | B | A | B | B |
| | Rinsability | A | A | B | A | A | A | A | A | B |

| | | Comparative | Comparative | Comparative | Comparative | Comparative | Comparative | Comparative | Comparative | Comparative | Comparative | Comparative | Comparative |
|---|---|---|---|---|---|---|---|---|---|---|---|---|---|
| | | Example 13 | Example 14 | Example 15 | Example 16 | Example 17 | Example 18 | Example 19 | Example 20 | Example 21 | Example 22 | Example 23 | Example 24 |
| Component A: | sodium cocoyl | 6.00 | 6.00 | 6.00 | 15.00 | 15.00 | 15.00 | 7.50 | 7.50 | 9.00 | 9.00 | 12.00 | 12.00 |
| | glycinate | | | | | | | | | | | | |
| Component B: | sodium | 10.00 | 8.00 | 6.00 | 2.50 | 2.00 | 1.50 | 10.50 | 3.50 | 9.00 | 3.00 | 6.00 | 2.00 |
| | polyoxyethylene | | | | | | | | | | | | |
| | lauryl ether | | | | | | | | | | | | |
| | sulfate | | | | | | | | | | | | |
| Component C: | cocamidopropyl | 2.00 | 4.00 | 6.00 | 0.50 | 1.00 | 1.50 | 0.00 | 7.00 | 0.00 | 6.00 | 0.00 | 4.00 |
| | betaine | | | | | | | | | | | | |
| Component D: | sodium chloride | 0.5 | 0.5 | 0.5 | 0.5 | 0.5 | 0.5 | 0.5 | 0.5 | 0.5 | 0.5 | 0.5 | 0.5 |
| | citric acid | pH 5.5 | pH 5.5 | pH 5.5 | pH 5.5 | pH 5.5 | pH 5.5 | pH 5.5 | pH 5.5 | pH 5.5 | pH 5.5 | pH 5.5 | pH 5.5 |
| | monohydrate | | | | | | | | | | | | |
| Component E: | water | balance | balance | balance | balance | balance | balance | balance | balance | balance | balance | balance | balance |
| | Total | 100.0 | 100.0 | 100.0 | 100.0 | 100.0 | 100.0 | 100.0 | 100.0 | 100.0 | 100.0 | 100.0 | 100.0 |

| Component A/(Component A + Component | | 1/3 | 1/3 | 1/3 | 5/6 | 5/6 | 5/6 | 5/12 | 5/12 | 1/2 | 1/2 | 2/3 | 2/3 |
|---|---|---|---|---|---|---|---|---|---|---|---|---|---|
| B + Component C) | | | | | | | | | | | | | |
| Component B/Component C | | 5/1 | 2/1 | 1/1 | 5/1 | 2/1 | 1/1 | 1/0 | 1/2 | 1/0 | 1/2 | 1/0 | 1/2 |
| Primary | Form | L | V | L | P | P | P | P | P | P | P | P | P |
| Evaluation: | Decision | C | B | C | A | A | A | A | A | A | A | A | A |
| Secondary | Whiteness | — | B | D | A | A | A | C | B | B | C | B | B |
| Evaluation: | Softness | — | C | D | C | C | C | B | A | C | B | D | A |
| | Smoothness | — | D | D | C | C | C | D | B | D | D | C | B |
| | Break | — | A | D | A | A | A | D | C | B | C | D | C |
| | Softness (0° C.) | — | D | D | D | D | D | B | A | D | B | D | A |
| Tertiary | Foamability | — | — | D | D | D | D | — | D | — | D | — | D |
| Evaluation: | Rinsability | — | — | D | A | A | B | — | D | — | — | — | D |

| | | Example 19 | Example 20 | Example 21 | Example 22 | Example 23 | Example 24 | Example 25 | Example 26 | Example 27 |
|---|---|---|---|---|---|---|---|---|---|---|
| Comonent A: | sodium cocoyl glycinate | 7.50 | 7.50 | 7.50 | 9.00 | 9.00 | 9.00 | 12.00 | 12.00 | 12.00 |
| Component B: | sodium polyoxyethylene | 8.75 | 7.00 | 5.25 | 7.50 | 6.00 | 4.50 | 5.00 | 4.00 | 3.00 |
| | lauryl ether sulfate | | | | | | | | | |
| Component C: | cocamidopropyl betaine | 1.75 | 3.50 | 5.25 | 1.50 | 3.00 | 4.50 | 1.00 | 2.00 | 3.00 |
| Component D: | sodium chloride | 0.5 | 0.5 | 0.5 | 0.5 | 0.5 | 0.5 | 0.5 | 0.5 | 0.5 |
| | citric acid monohydrate | pH 5.0 | pH 5.0 | pH 5.0 | pH 5.0 | pH 5.0 | pH 5.0 | pH 5.0 | pH 5.0 | pH 5.0 |
| Component E: | water | balance | balance | balance | balance | balance | balance | balance | balance | balance |
| | Total | 100.0 | 100.0 | 100.0 | 100.0 | 100.0 | 100.0 | 100.0 | 100.0 | 100.0 |

| Component A/(Component A + Component | | 5/12 | 5/12 | 5/12 | 1/2 | 1/2 | 1/2 | 2/3 | 2/3 | 2/3 |
|---|---|---|---|---|---|---|---|---|---|---|
| B + Component C) | | | | | | | | | | |
| Component B/Component C | | 5/1 | 2/1 | 1/1 | 5/1 | 2/1 | 1/1 | 5/1 | 2/1 | 1/1 |
| Primary | Form | P | P | P | P | P | P | P | P | P |
| Evaluation: | Decision | A | A | A | A | A | A | A | A | A |
| Secondary | Whiteness | A | A | B | A | A | A | B | A | A |
| Evaluation: | Softness | A | A | A | A | A | A | A | A | A |
| | Smoothness | A | A | A | B | B | B | B | B | B |
| | Break | A | A | B | A | A | A | A | A | A |
| | Softness (0° C.) | A | A | A | B | B | B | B | B | B |
| Tertiary | Foamability | A | A | A | A | B | B | B | B | B |
| Evaluation: | Rinsability | A | A | B | A | A | A | A | A | B |

| | | Comparative | Comparative | Comparative | Comparative | Comparative | Comparative | Comparative | Comparative | Comparative | Comparative | Comparative | Comparative |
|---|---|---|---|---|---|---|---|---|---|---|---|---|---|
| | | Example 25 | Example 26 | Example 27 | Example 28 | Example 29 | Example 30 | Example 31 | Example 32 | Example 33 | Example 34 | Example 35 | Example 36 |
| Component A: | sodium cocoyl | 6.00 | 6.00 | 6.00 | 15.00 | 15.00 | 15.00 | 7.50 | 7.50 | 9.00 | 9.00 | 12.00 | 12.00 |
| | glycinate | | | | | | | | | | | | |
| Component B: | sodium | 10.00 | 8.00 | 6.00 | 2.50 | 2.00 | 1.50 | 10.50 | 3.50 | 9.00 | 3.00 | 6.00 | 2.00 |
| | polyoxyethylene | | | | | | | | | | | | |
| | lauryl ether sulfate | | | | | | | | | | | | |
| Component C: | cocamidopropyl | 2.00 | 4.00 | 6.00 | 0.50 | 1.00 | 1.50 | 0.00 | 7.00 | 0.00 | 6.00 | 0.00 | 4.00 |
| | betaine | | | | | | | | | | | | |
| Component D: | sodium chloride | 0.5 | 0.5 | 0.5 | 0.5 | 0.5 | 0.5 | 0.5 | 0.5 | 0.5 | 0.5 | 0.5 | 0.5 |
| | citric acid | pH 5.0 | pH 5.0 | pH 5.0 | pH 5.0 | pH 5.0 | pH 5.0 | pH 5.0 | pH 5.0 | pH 5.0 | pH 5.0 | pH 5.0 | pH 5.0 |
| | monohydrate | | | | | | | | | | | | |
| Component E: | water | balance | balance | balance | balance | balance | balance | balance | balance | balance | balance | balance | balance |
| | Total | 100.0 | 100.0 | 100.0 | 100.0 | 100.0 | 100.0 | 100.0 | 100.0 | 100.0 | 100.0 | 100.0 | 100.0 |

| Component A/(Component A + Component | | 1/3 | 1/3 | 1/3 | 5/6 | 5/6 | 5/6 | 5/12 | 5/12 | 1/2 | 1/2 | 2/3 | 2/3 |
|---|---|---|---|---|---|---|---|---|---|---|---|---|---|
| B + Component C) | | | | | | | | | | | | | |
| Component B/Component C | | 5/1 | 2/1 | 1/1 | 5/1 | 2/1 | 1/1 | 1/0 | 1/2 | 1/0 | 1/2 | 1/0 | 1/2 |
| Primary | Form | L | V | V | S | S | S | V | P | P | P | P | P |
| Evaluation: | Decision | C | B | B | B | B | B | B | A | A | A | A | A |
| Secondary | Whiteness | — | B | B | A | A | A | B | B | B | B | B | B |
| Evaluation: | Softness | — | C | C | D | D | D | C | A | C | A | D | A |
| | Smoothness | — | D | D | D | D | D | D | B | D | D | C | D |
| | Break | — | A | C | A | A | A | A | C | B | C | D | C |
| | Softness (0° C.) | — | D | D | D | D | D | D | A | D | C | D | C |
| Tertiary | Foamability | — | — | — | — | — | — | — | D | — | D | — | D |
| Evaluation: | Rinsability | — | — | — | — | — | — | — | D | — | — | — | — |

| | | Comparative | Comparative | Comparative | Comparative | Comparative | Comparative | Comparative | Comparative | Comparative |
|---|---|---|---|---|---|---|---|---|---|---|
| | | Examlpe 37 | Examlpe 38 | Examlpe 39 | Example 40 | Examlpe 41 | Examlpe 42 | Examlpe 43 | Examlpe 44 | Examlpe 45 |
| Component A: | sodium cocoyl | 7.50 | 7.50 | 7.50 | 9.00 | 9.00 | 9.00 | 12.00 | 12.00 | 12.00 |
| | glycinate | | | | | | | | | |
| Component B: | sodium | 8.75 | 7.00 | 5.25 | 7.50 | 6.00 | 4.50 | 5.00 | 4.00 | 3.00 |
| | polyoxyethylene | | | | | | | | | |
| | lauryl ether sulfate | | | | | | | | | |
| Component C: | cocamidopropyl | 1.75 | 3.50 | 5.25 | 1.50 | 3.00 | 4.50 | 1.00 | 2.00 | 3.00 |
| | betaine | | | | | | | | | |
| Component D: | sodium chloride | 0.5 | 0.5 | 0.5 | 0.5 | 0.5 | 0.5 | 0.5 | 0.5 | 0.5 |
| | citric acid | pH 6.5 | pH 6.5 | pH 6.5 | pH 6.5 | pH 6.5 | pH 6.5 | pH 6.5 | pH 6.5 | pH 6.5 |
| | monohydrate | | | | | | | | | |
| Component E: | water | balance | balance | balance | balance | balance | balance | balance | balance | balance |
| | Total | 100.0 | 100.0 | 100.0 | 100.0 | 100.0 | 100.0 | 100.0 | 100.0 | 100.0 |

| Component A/(Component A + Component | | 5/12 | 5/12 | 5/12 | 1/2 | 1/2 | 1/2 | 2/3 | 2/3 | 2/3 |
|---|---|---|---|---|---|---|---|---|---|---|
| B + Component C) | | | | | | | | | | |
| Component B/Component C | | 5/1 | 2/1 | 1/1 | 5/1 | 2/1 | 1/1 | 5/1 | 2/1 | 1/1 |
| Primary | Form | L | L | L | L | L | L | L | L | L |
| Evaluation: | Decision | C | C | C | C | C | C | C | C | C |
| Secondary | Whiteness | — | — | — | — | — | — | — | — | — |
| Evaluation: | Softness | — | — | — | — | — | — | — | — | — |
| | Smoothness | — | — | — | — | — | — | — | — | — |
| | Break | — | — | — | — | — | — | — | — | — |
| | Softness (0° C.) | — | — | — | — | — | — | — | — | — |
| Tertiary | Foamability | — | — | — | — | — | — | — | — | — |
| Evaluation: | Rinsability | — | — | — | — | — | — | — | — | — |

| | | Comparative | Comparative | Comparative | Comparative | Comparative | Comparative | Comparative | Comparative | Comparative |
|---|---|---|---|---|---|---|---|---|---|---|
| | | Examlpe 46 | Examlpe 47 | Examlpe 48 | Examlpe 49 | Examlpe 50 | Examlpe 51 | Example 52 | Examlpe 53 | Example 54 |
| Component A: | sodium cocoyl glycinate | 7.50 | 7.50 | 7.50 | 9.00 | 9.00 | 9.00 | 12.00 | 12.00 | 12.00 |
| Component B: | sodium | 8.75 | 7.00 | 5.25 | 7.50 | 6.00 | 4.50 | 5.00 | 4.00 | 3.00 |
| | polyoxyethylene lauryl | | | | | | | | | |
| | ether sulfate | | | | | | | | | |
| Component C: | cocamidopropyl betaine | 1.75 | 3.50 | 5.25 | 1.50 | 3.00 | 4.50 | 1.00 | 2.00 | 3.00 |
| Component D: | sodium chloride | 0.5 | 0.5 | 0.5 | 0.5 | 0.5 | 0.5 | 0.5 | 0.5 | 0.5 |
| | citric acid monohydrate | pH 4.8 | pH 4.8 | pH 4.8 | pH 4.8 | pH 4.8 | pH 4.8 | pH 4.8 | pH 4.8 | pH 4.8 |
| Component E: | water | balance | balance | balance | balance | balance | balance | balance | balance | balance |
| | Total | 100.0 | 100.0 | 100.0 | 100.0 | 100.0 | 100.0 | 100.0 | 100.0 | 100.0 |

| Component A/(Component A + Component | | 5/12 | 5/12 | 5/12 | 1/2 | 1/2 | 1/2 | 2/3 | 2/3 | 2/3 |
|---|---|---|---|---|---|---|---|---|---|---|
| B + Component C) | | | | | | | | | | |
| Component B/Component C | | 5/1 | 2/1 | 1/1 | 5/1 | 2/1 | 1/1 | 5/1 | 2/1 | 1/1 |
| Primary Evaluation: | Form | P | P | P | P | P | P | P | P | P |
| | Decision | A | A | A | A | A | A | A | A | A |
| Secondary | Softness | A | A | A | A | A | A | A | A | A |
| Evaluation: | Smoothness | C | C | C | C | C | C | C | C | C |
| | Break | B | B | B | B | B | B | C | C | C |
| | Softness (0° C.) | B | B | B | B | B | B | C | C | C |
| | Softness | D | D | D | D | D | D | D | D | D |
| Tertiary Evaluation: | Foambility | D | D | D | D | D | D | D | D | D |
| | Rinsability | — | — | — | — | — | — | — | — | — |

As is seen from Table 1, the cleansing compositions of Examples exhibited a creamy form, and the cream had strong whiteness, was soft and smooth with favorable break, without getting hard at a low temperature, and was excellent in foaming and rinsing properties.

### Examples 28 to 29, Comparative Examples 55 to 64

Cleansing compositions (Examples 28 and 29, Comparative Examples 55 to 64) having the composition shown in Table 2 below were prepared, and tested according to the following procedures The amount of each compounded component shown in the Table (values in Table) represents the weight fraction (%) provided that total composition accounts for 100.

### Procedure

1. Primary Evaluation: Evaluation of Composition Form (Form) was evaluated.

2. Secondary Evaluation: Evaluation of Quality of Composition as Cream

### The compositions determined as A or B in the primary evaluation were evaluated on the (Odor), (Whiteness), (Smoothness), (Break), and (Blendability with Water).

3. Tertiary Evaluation

### Evaluation on the (Foamability) and (Rinsability) was made for the compositions except for those categorized into D on two or more items in the secondary evaluation.

4. Quaternary Evaluation: Ease in Preparation of Composition and Softness

### Only the compositions evaluated to fall in the category P in the primary evaluation were evaluated on the (Air Bubble Entrainment Height), (Hardness), and (Softness (0° C.)).

TABLE 2

| | | | | Patent | Patent | Patent | Patent | Patent |
|---|---|---|---|---|---|---|---|---|
| | | | | Document 1 | Document 1 | Document 2 | Document 3 | Document 3 |
| | | Example | | Comparative | Comparative | Comparative | Comparative | Comparative |
| | | 28 | Example 29 | Example 55 | Example 56 | Example 57 | Example 58 | Example 59 |
| Component A: | sodium cocoyl glycinate | 7.5 | — | — | — | 20.0 | 30.0 | 30.0 |
| | potassium cocoyl glycinate | — | 12.0 | 27.0 | 20.0 | — | — | — |
| Component B: | sodium polyoxyethylene | 5.3 | 5.0 | — | — | — | — | — |
| | lauryl ether sulfate | | | | | | | |
| Component C: | coconut oil fatty amidopropyl | 5.3 | 1.0 | — | — | — | — | 5.0 |
| | betaine | | | | | | | |
| | 2-alkyl-N-carboxymethyl-N- | — | — | — | — | — | — | — |
| | hydroxyethylimidazolinium betaine | | | | | | | |
| | coco-betaine | — | — | — | — | — | 30.0 | — |
| Component D: | sodium chloride | — | 0.5 | 10.0 | 12.0 | — | — | — |
| | sodium L-pyrrolidone | 1.0 | — | — | — | — | — | — |
| | carboxylate | | | | | | | |
| | sodium lauryl sarcosinate | — | — | — | — | — | — | — |
| | glycerin | — | — | — | — | 40.0 | 30.0 | — |
| | butylene glycol | — | — | — | 15.0 | — | — | |
| | propylene glycol | — | — | — | | | 5.0 | — |
| | citric acid monohydrate | 0.4 | 1.1 | — | — | 1.0 | 1.0 | 2.4 |
| | O-[2-hydroxy-3- | — | — | — | — | — | — | — |
| | (trimethylammonio)propyl]chloride | | | | | | | |
| | sodium lauryl ether carboxylate | — | — | — | — | — | — | — |
| | lauric acid diethanol amide | 2.0 | — | — | — | — | — | — |
| | decyl glucoside | — | 2.0 | — | — | — | — | — |
| | monoglyceryl laurate | — | — | — | — | — | — | |
| | bentonite | — | — | — | — | — | — | — |
| | silicic anhydride | — | — | — | — | — | — | — |
| | polyoxyethylene glycol | — | — | — | — | 0.2 | 0.2 | — |
| | (average molecular | | | | | | | |
| | weight 70,000) | | | | | | | |
| | polyoxyethylene (200) | — | — | — | — | — | — | — |
| | polypropylene (44)glycol | | | | | | | |
| Component E: | water | balance | balance | balance | balance | balance | balance | balance |
| | Total | 100.0 | 100.0 | 100.0 | 100.0 | 100.0 | 100.0 | 100.0 |
| Primary | Form | P | P | V | SP | P | P | P |
| Evaluation | Decision | A | A | B | C | A | A | A |
| Secondary | Odor | B | B | B | — | C | D | B |
| Evaluation | Whiteness | B | A | D | — | B | C | B |
| | Smoothness | A | A | A | — | B | D | D |
| | Break | A | A | C | — | A | A | A |
| | Blendability with Water | A | A | D | — | B | B | B |
| Tertiary | Foamability | A | A | — | — | — | — | C |
| Evaluation | Rinsability | A | A | — | — | — | — | C |
| Quaternary | Air Bubble Entrainment | 3.8 | 2.5 | — | — | 8.5 | 7.0 | 8.0 |
| Evaluation | Height (cm) | | | | | | | |
| | Decision | B | A | — | — | D | D | D |
| | Hardness (g) | 20 | 16 | — | — | 621 | 814 | 243 |
| | Decision | A | A | — | — | D | D | C |
| | Softness (0° C.) | A | A | — | — | D | D | B |

| | | Patent | Patent | Patent | Patent | Patent |
|---|---|---|---|---|---|---|
| | | Document 4 | Document 5 | Document 5 | Document 6 | Document 6 |
| | | Comparative | Comparative | Comparative | Comparative | Comparative |
| | | Example 60 | Example 61 | Example 62 | Example 63 | Example 64 |
| Component A: | sodium cocoyl glycinate | — | — | 5.0 | — | — |
| | potassium cocoyl glycinate | 28.0 | 9.0 | 3.0 | — | — |
| Component B: | sodium polyoxyethylene | — | — | 0.8 | 10.00 | 8.75 |
| | lauryl ether sulfate | | | | | |
| Component C: | coconut oil fatty amidopropyl | — | — | — | 5.00 | 1.75 |
| | betaine | | | | | |
| | 2-alkyl-N-carboxymethyl-N- | 2.0 | — | 1.5 | — | — |
| | hydroxyethylimidazolinium betaine | | | | | |
| | coco-betaine | — | 15.0 | 5.0 | — | — |
| Component D: | sodium chloride | — | — | — | 2.00 | 0.30 |
| | sodium L-pyrrolidone | — | — | — | — | — |
| | carboxylate | | | | | |
| | sodium lauryl sarcosinate | — | — | — | 4.50 | 7.50 |
| | glycerin | — | — | — | — | — |
| | butylene glycol | 37.6 | — | — | — | — |
| | propylene glycol | | 12.0 | — | — | — |
| | citric acid monohydrate | 2.4 | — | — | 0.20 | pH 5.5 |
| | O-[2-hydroxy-3- | — | — | 2.0 | — | — |
| | (trimethylammonio)propyl]chloride | | | | | |
| | sodium lauryl ether carboxylate | — | | 3.0 | — | — |
| | lauric acid diethanol amide | — | 5.0 | — | — | 2.00 |
| | decyl glucoside | — | — | — | — | — |
| | monoglyceryl laurate | 1.0 | — | — | — | — |
| | bentonite | 0.1 | — | — | — | — |
| | silicic anhydride | — | 8.0 | 10.0 | — | — |
| | polyoxyethylene glycol | — | — | — | — | — |
| | (average molecular | | | | | |
| | weight 70,000) | | | | | |
| | polyoxyethylene (200) | — | 1.0 | 2.5 | — | — |
| | polypropylene (44)glycol | | | | | |
| Component E: | water | balance | balance | balance | balance | balance |
| | Total | 100.0 | 100.0 | 100.0 | 100.0 | 100.0 |
| Primary | Form | P | SP | SP | V | V |
| Evaluation | Decision | A | C | C | B | B |
| Secondary | Odor | D | — | — | B | B |
| Evaluation | Whiteness | A | — | — | D | D |
| | Smoothness | D | — | — | A | A |
| | Break | A | — | — | D | D |
| | Blendability with Water | C | — | — | D | D |
| Tertiary | Foamability | — | — | — | — | — |
| Evaluation | Rinsability | — | — | — | — | — |
| Quaternary | Air Bubble Entrainment | 7.0 | — | — | — | — |
| Evaluation | Height (cm) | | | | | |
| | Decision | D | — | — | — | — |
| | Hardness (g) | 1134 | — | — | — | — |
| | Decision | D | — | — | — | — |
| | Softness (0° C.) | D | — | — | — | — |

As is seen from Table 2, in the composition in which silicic anhydride was blended as the component A, solid-liquid separation was caused by sedimentation of silicic anhydride (Comparative Examples 61, 62 (corresponding to cited document 5)). The composition in which the component B, the component C, the component D and the component E were blended with acyl sarcosine that is an analogue of the component A was too viscous, and was inferior in all terms of whiteness, break and blendability with water (Comparative Examples 63, 64 (corresponding to cited document 6)). Also, in the case of the composition in which the component D (inorganic salt) was blended with the component A, insufficient whiteness, blendability with water could be achieved although a viscous composition could be prepared (Comparative Examples 55, 56 (corresponding to cited document 1)). The composition in which polyhydric alcohol was blended with the component A exhibited high air bubble entrainment, and in addition, became unusually hard (Comparative Example 57 (corresponding to cited document 2), Comparative Example 58 (corresponding to cited document 3)). In the composition in which the component C (betaine amphoteric surfactant) was blended with the component A, great air bubble entrainment was exhibited, and moreover, insufficient smoothness was achieved (Comparative Example 59 (corresponding to cited document 3), Comparative Example 60 (corresponding to cited document 4)). Accordingly, even though tracing experiments were carried out in connection with any cited documents, they were far away from providing cream-type compositions. To the contrary, it was proven that the composition of the invention exhibited a creamy form having strong whiteness, being soft and smooth with favorable break, without getting hard at a low temperature, and air bubble entrainment hardly occurred during the production, accompanied by excellent foaming and rinsabilities (Examples 28, 29).

### Examples 30 to 32, Comparative Examples 65 to 76

Cleansing compositions (Examples 30 to 32, Comparative Examples 65 to 76) having the composition shown in Table 3 below were prepared, and tested according to the following procedures The amount of each compounded component shown in the Table (values in Table) represents the weight fraction (%) provided that total composition accounts for 100. Also, citric acid monohydrate was used each in an amount required for adjusting the pH of the composition.

### Procedure

1. Primary Evaluation: Evaluation of Composition Form (Form) was evaluated.

2. Secondary Evaluation: Evaluation of Quality of Composition as Cream

### The compositions determined as A or B in the primary evaluation were evaluated on the (Whiteness), (Softness), (Smoothness), (Break), and (Blendability with Water).

TABLE 3

| | | | | | | | | | Compar- | Compar- |
|---|---|---|---|---|---|---|---|---|---|---|
| | | | | | | | | | ative | ative |
| | | Example | Example | | Comparative | Comparative | Comparative | Comparative | Exam- | Exam- |
| | | 30 | 31 | Example 32 | Example 65 | Example 66 | Example 67 | Example 68 | ple 69 | ple 70 |
| Compo- | potassium | 7.50 | 9.00 | 12.00 | — | — | — | — | — | — |
| nent A: | cocoyl | | | | | | | | | |
| | glycinate | | | | | | | | | |
| | sodium | — | — | — | 7.50 | 9.00 | 12.00 | 7.50 | 9.00 | 12.00 |
| | cocoyl | | | | | | | | | |
| | alaninate | | | | | | | | | |
| | sodium | — | — | — | — | — | — | — | — | — |
| | laurate | | | | | | | | | |
| Compo- | sodium | 8.75 | 7.50 | 5.00 | 8.75 | 7.50 | 5.00 | 8.75 | 7.50 | 5.00 |
| nent B: | polyoxyethylene | | | | | | | | | |
| | lauryl ether | | | | | | | | | |
| | sulfate | | | | | | | | | |
| Compo- | cocamidopropyl | 1.75 | 1.50 | 1.00 | 1.75 | 1.50 | 1.00 | 1.75 | 1.50 | 1.00 |
| nent C: | betaine | | | | | | | | | |
| | citric acid | pH 6.0 | pH 6.0 | pH 6.0 | pH 5.5 | pH 5.5 | pH 5.5 | pH 5.0 | pH 5.0 | pH 5.0 |
| | monohydrate | | | | | | | | | |
| Compo- | sodium | 0.30 | 0.30 | 0.30 | 0.30 | 0.30 | 0.30 | 0.30 | 0.30 | 0.30 |
| nent D: | chloride | | | | | | | | | |
| | lauric acid | 2.00 | 2.00 | 2.00 | 2.00 | 2.00 | 2.00 | 2.00 | 2.00 | 2.00 |
| | diethanol amide | | | | | | | | | |
| Compo- | water | balance | balance | balance | balance | balance | balance | balance | balance | balance |
| nent E: | | | | | | | | | | |
| | Total | 100.0 | 100.0 | 100.0 | 100.0 | 100.0 | 100.0 | 100.0 | 100.0 | 100.0 |
| Primary | Form | P | P | P | L | L | L | V | L | L |
| Evaluation | Decision | A | A | A | C | C | C | B | C | C |
| Secondary | Whiteness | B | B | B | — | — | — | D | — | — |
| Evaluation | Softness | A | A | A | — | — | — | A | — | — |
| | Smoothness | A | A | B | — | — | — | A | — | — |
| | Break | B | A | A | — | — | — | C | — | — |
| | Blendability | A | A | A | — | — | — | D | — | — |
| | with Water | | | | | | | | | |

| | | Comparative | Comparative | Comparative | Comparative | Comparative | Comparative |
|---|---|---|---|---|---|---|---|
| | | Example 71 | Example 72 | Example 73 | Example 74 | Example 75 | Example 76 |
| Component A: | potassium cocoyl glycinate | — | — | — | — | — | — |
| | sodium cocoyl alaninate | — | — | — | — | — | — |
| | sodium laurate | 7.50 | 9.00 | 12.00 | 7.50 | 9.00 | 12.00 |
| Component B: | sodium polyoxyethylene | 8.75 | 7.50 | 5.00 | 8.75 | 7.50 | 5.00 |
| | lauryl ether sulfate | | | | | | |
| Component C: | cocamidopropyl betaine | 1.75 | 1.50 | 1.00 | 1.75 | 1.50 | 1.00 |
| | citric acid monohydrate | pH 6.0 | pH 6.0 | pH 6.0 | pH 5.5 | pH 5.5 | pH 5.5 |
| Component D: | sodium chloride | 0.30 | 0.30 | 0.30 | 0.30 | 0.30 | 0.30 |
| | lauric acid diethanol amide | 2.00 | 2.00 | 2.00 | 2.00 | 2.00 | 2.00 |
| Component E: | water | balance | balance | balance | balance | balance | balance |
| | Total | 100.00 | 100.00 | 100.00 | 100.00 | 100.00 | 100.00 |
| Primary Evaluation | Form | L | L | L | L | L | L |
| | Decision | C | C | C | C | C | C |
| Secondary Evaluation | Whiteness | — | — | — | — | — | — |
| | Softness | — | — | — | — | — | — |
| | Smoothness | — | — | — | — | — | — |
| | Break | — | — | — | — | — | — |
| | Blendability with Water | — | — | — | — | — | — |

As is seen from Table 3, the cleansing compositions of Examples exhibited a creamy form having strong whiteness, and favorable blendability with water.

### Examples 33 to 35

Cleansing compositions (Examples 33 to 36) having the composition shown in Table 4 below were prepared, and evaluation on the (Whiteness), (Softness), (Smoothness), and (Stability at High Temperature) was made. The amount of each compounded component shown in the Table (values in Table) represents the weight fraction (%) provided that total composition accounts for 100. Also, citric acid monohydrate was used each in an amount required for adjusting the pH of the composition.

TABLE 4

| | | Example 33 | Example 34 | Example 35 | Example 36 |
|---|---|---|---|---|---|
| Component A: | sodium cocoyl glycinate | 16.00 | 16.00 | 16.00 | 16.00 |
| Component B: | sodium polyoxyethylene | 6.67 | 6.67 | 6.67 | 6.67 |
| | lauryl ether sulfate | | | | |
| Component C: | cocamidopropyl betaine | 1.33 | 1.33 | 1.33 | 1.33 |
| | citric acid monohydrate | pH 5.8 | pH 5.8 | pH 5.8 | pH 5.8 |
| Component D: | sodium chloride | 0.5 | 0.30 | 0.5 | 0.30 |
| | lauric acid diethanol amide | 2.00 | 2.00 | 2.00 | 2.00 |
| Component F: | lauric acid | — | 0.50 | — | 0.50 |
| Component G: | lauryl glucoside | — | — | 1.00 | 1.00 |
| Component E: | water | balance | balance | balance | balance |
| | Total | 100.00 | 100.00 | 100.00 | 100.00 |
| Evaluation | Whiteness | B | A | B | A |
| | Softness | A | A | A | A |
| | Smoothness | B | A | B | A |
| | Stability at high temperature | C | C | A | A |

As is seen from Table 4, when a fatty acid was further combined with the cleansing composition of Example 33, the whiteness and smoothness was increased (Example 34), and addition of alkylpolyglucoside accomplished surprising improvement of the stability at a high temperature (Example 35). Furthermore, it was revealed that surprising improvement of the stability at a high temperature could be achieved without adversely affecting the softness and smoothness of the cream in the case in which both were used in combination (Example 36).

### INDUSTRIAL APPLICABILITY

It is extremely advantageous that cream-type skin cleansing compositions, various cleansing agents and cosmetics containing substantially no polyhydric alcohol, being weakly acidic with a pH of 4.0 to 6.0, and soft and smooth with a whity appearance can be provided, which are excellent in foamability and rinsability and in feeling during application such as moisturizing and fresh dry-skin feel, but can avoid from getting hard at low temperatures, and which can be readily prepared by using a common simple mixer.

The present application is based on Japanese Application No. 2006-095878, filed on Mar. 30, 2006 and incorporated herein by reference.

1. A cream-type skin cleansing composition comprising:
an N-long-chain acylglycine and/or a salt thereof (component A),
a polyoxyethylene alkyl ether sulfate salt (component B),
a betaine type amphoteric surfactant (component C),
an inorganic salt and/or a pyrrolidone carboxylate salt (component D), and
water (component E),
wherein
the (total weight of the (component A), (component B) and (component C)) is 12 to 30% by weight of total weight of the composition;
the (weight of the (component A))/(total weight of the (component A)), (component B) and (component C)) is 3/8 to 4/5;
the (weight of the (component B))/(weight of the (component C)) is 9/1 to 1/1; and
the pH being 4.0 to 6.0.

2. The composition of claim 1, which further comprises a higher fatty acid (component F) and/or alkylpolyglucoside (component G) in an amount of 0.01 to 5.0% by weight.

3. The composition of claim 1, which contains substantially no polyhydric alcohol.

4. The composition of claim 1, which is free of polyhydric alcohol.

5. The composition of claim 1, wherein the long-chain acyl group of the long-chain N-acylglycine and/or the salt thereof is derived from a saturated or unsaturated fatty acid having a straight or branched chain with 8 to 22 carbon atoms can be used.

6. The composition of claim 1, wherein the long-chain acyl group is derived from caprylic acid, capric acid, lauric acid, myristic acid, stearic acid, isostearic acid, palmitic acid, oleic acid, linoleic acid, behenic acid, coconut oil fatty acid, palm fatty acid, or hydrogenated beef tallow fatty acid,

7. The composition of claim 1, which contains a salt of an N-long-chain acylglycine selected from the group consisting of alkali metal salts, alkaline earth metal salts, organic amine salts, basic amino acid salts and mixtures thereof.

8. The composition of claim 1, wherein the polyoxyethylene alkyl ether sulfate salt is a lauryl ether sulfate salt.

9. The composition of claim 1, wherein the polyoxyethylene alkyl ether sulfate salt is represented by the formula:
R—(OC₂H₄)ₙ—OSO₃M (1)
wherein
R represents an alkyl group having 8 to 24 carbon atoms;
n represents an average number of added moles of ethylene oxide, and M represents a metal, organic amine, or a basic amino acid.

10. The composition of claim 1, wherein the polyoxyethylene alkyl ether sulfate salt is an alkali metal salt, an alkaline earth metal salt, an organic amine salt, or a basic amino acid salt.

11. The composition of claim 1, wherein the betaine type amphoteric surfactant is an acetic betaine type amphoteric surfactant, an amide betaine type amphoteric surfactant, a sulfo betaine type amphoteric surfactant, a phospho betaine type amphoteric surfactant, or an imidazolinium betaine type amphoteric surfactant.

12. The composition of claim 1, wherein the betaine type amphoteric surfactant is represented by the formula: 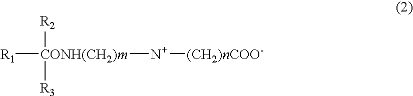
wherein
R¹ represents a straight or branched alkyl or alkenyl group having 5 to 21 carbon atoms;
R², R³, independently, represent a hydrogen atom, or a straight or branched alkyl group having 1 to 3 carbon atoms; and
n represents an integer of 1 to 3; and m represents an integer of 1 to 4.

13. The composition of claim 1, wherein component D comprises the inorganic salt and, wherein the inorganic salt is sodium chloride, potassium chloride, magnesium chloride, calcium chloride, sodium sulfate, sodium hydrogen sulfate, potassium sulfate, potassium hydrogen sulfate, magnesium sulfate, calcium sulfate, monosodium phosphate, disodium phosphate, monopotassium phosphate, dipotassium phosphate, sodium carbonate, disodium hydrogen carbonate, potassium carbonate, potassium hydrogen carbonate, magnesium carbonate, calcium carbonate, sodium pyrrolidone carboxylate, sodium L-pyrrolidone carboxylate, potassium DL-pyrrolidone carboxylate, or ammonium DL-pyrrolidone carboxylate.

14. The composition of claim 1, wherein component D comprises the pyrrolidone carboxylate, wherein the pyrrolidone carboxylate is sodium DL-pyrrolidone carboxylate, sodium L-pyrrolidone carboxylate, or potassium DL-pyrrolidone carboxylate

15. The composition of claim 1, which has a pH of 5.3 to 5.8.

16. The composition of claim 1, wherein the total weight of the (component A), (component B) and (component C) is 16 to 25% by weight.

17. The composition of claim 1, wherein the ratio of (weight of the (component A))/(total weight of the (component A), (component B) and (component C)) is 5/12 to 2/3.

18. The composition of claim 1, wherein the ratio (weight of the (component B))/(weight of the (component C)) is 3/1 to 5/1.

19. A method of preparing the composition of claim 1, comprising combining component A, component B, component C, component D and component E.

20. A method of treating skin, comprising applying the composition of claim 1 to the skin.
